(19) United States
(12) Patent Application Publication
(10) Pub. No.: US 2022/0133373 A1
(43) Pub. Date: May 5, 2022

(54) ENHANCED ORTHOPEDIC SCREW

(71) Applicant: Peter STEVENS, Salt Lake City, UT (US)

(72) Inventor: Peter STEVENS, Salt Lake City, UT (US)

(21) Appl. No.: 17/429,025

(22) PCT Filed: Feb. 10, 2020
(86) PCT No.: PCT/US2020/017426

§ 371 Date: Aug. 6, 2021

Related U.S. Application Data
(60) Provisional application No. 62/802,868, filed on Feb. 8, 2019.

(51) Int. Cl. A61B 17/86; A61F 2/42

(52) U.S. Cl. CPC A61B 17/862; A61B 17/863; A61F 2002/4223; A61F 2/4202; A61B 17/8635

(57) ABSTRACT

A subtalar arthroereisis screw may have a head and a body with a distal tip, a shank extending from the head to the distal tip, and a screw thread extending along the shank. The shank may have a leading end proximate the distal tip and a trailing end proximate the head. The screw thread may be continuously variable between a first pitch relatively closer to the trailing end, and a second pitch, larger than the first pitch, relatively closer to the leading end. The head may have a receiving end that is connectable to a tool to facilitate rotation of the head with the tool during implantation of the subtalar arthroereisis screw. The screw thread may taper continuously from a first major diameter at the trailing end, to a second major diameter, smaller than the first major diameter, and may have a plurality of notches distributed along its length.

### TECHNICAL FIELD

The present disclosure relates to surgical systems and methods. More specifically, the present disclosure relates to a screw that can be used in the correction of deformities of hones or muscles.

### BACKGROUND

A particular procedure known as Subtalar Extra-articular Screw Arthroereisis is used for the treatment of flexible flatfoot. Flexible flatfoot (hereafter as “FFF”) is a common skeletal disorder where a person has an abnormally low or absent arch, leading to an excessive eversion of the heel during weight-bearing, and an abducted forefoot producing midfoot sag. FFF also leads to other counter indications to the person's Achilles and can adversely affect a person's gait.

FFF is also thought to induce the progression of degenerative arthritis, leading to the loss of flexibility and ankylosis. An increasingly frequent pattern of tibialis posterior overuse has also been described as a result of FFF.

For symptomatic patients, inlays or even orthoses are sometimes recommended. However, recent literature on the effect of pediatric foot orthoses found very limited evidence on the effectiveness of non-surgical interventions with FFF.

Subtalar arthroereisis (also referred to as arthroisis) is the surgical implantation of a screw or similar implant to enable the limitation of movement across the subtalar joint. Subtalar arthroereisis or extraosseous talotarsal stabilization (EOTTS) corrects excessive talar displacement and SUR144 I 2 calcaneal eversion by reducing pronation across the subtalar joint. To fix FFF, a doctor places an implant, such as a screw, in the sinus tarsi, which is a canal located between the talus and the calcaneus. The subtalar implant acts as a spacer to block the anterior and inferior displacement of the talus, allowing normal subtalar joint motion but blocking excessive pronation and the resulting sequela. It has been performed for some 40 years with a variety of implant designs and compositions, primarily for treatment of flexible flatfoot, although the procedure and implant's interjection and use in other deformities, such as club foot have been reported. Subtalar arthroereisis is most often performed on young children and is designed to correct excessive talar displacement and calcaneal eversion.

Presently, the screws used as implants to correct FFF have the shortcoming of possible pre-mature withdrawal from the foot, or not providing a secure enough fit within the tissue for the implant to remain there and work for its intended purpose. Further, some known FFF implants may tend to form their own holes in soft tissue, making them difficult to guide once they have begun to penetrate tissue.

### SUMMARY

The various systems and methods of the present disclosure have been developed in response to the present state of the art, and in particular, in response to the problems and needs in the art that have not yet been fully solved by currently available subtalar arthroereisis systems and methods. The systems and methods of the present disclosure may provide subtalar arthroereisis implants, instruments, and methods that provide enhanced implant retention, streamlined implantation, and/or overall improved patient outcomes.

According to some embodiments, a subtalar arthroereisis screw may have a head and a body with a distal tip, a shank extending from the head to the distal tip, and a screw thread. The shank may have a leading end proximate the distal tip and a trailing end proximate the head. The screw thread may extend along at least a portion of the shank. The screw thread may be continuously variable between a first pitch relatively closer to the trailing end, and a second pitch, larger than the first pitch, relatively closer to the leading end. The head may have a receiving end that is connectable to a tool to facilitate rotation of the head with the tool during implantation of the subtalar arthroereisis screw.

The screw thread may taper continuously from a first major diameter at the trailing end, to a second major diameter, smaller than the first major diameter.

The screw thread may have a plurality of notches distributed along its length.

The distal tip may have a domelike shape.

The distal tip my have a generally hemispherical shape.

The head may be enlarged relative to the shank.

The receiving end of the head may have a drive feature that mates with a corresponding drive feature of the tool, and threading adjacent to the drive feature and matable with at least one selection from the group consisting of the tool, and a removal tool configured to remove the subtalar arthroereisis screw from an implantation site.

The subtalar arthroereisis screw may have a bore extending through the subtalar arthroereisis screw, from the head to the distal tip. The drive feature may have an interior feature defining a first portion of the bore, and the threading may be interior threading distal to the drive feature, defining a second portion of the bore.

The head may have a pair of cutouts positioned on opposite sides of the head. The cutouts may be shaped to facilitate gripping of the head with a removal tool.

The removal tool may be a Kocher clamp.

Each of the cutouts may have a concave surface flanked by two flattened surfaces.

According to one embodiment, a method for performing subtalar arthroereisis may include obtaining access to an implantation site in soft tissues of a foot and connecting a tool to a receiving end of a head of a subtalar arthroereisis screw. The subtalar arthroereisis screw may have the head and a body with a distal tip, a shank extending from the head to the distal tip, and a screw thread extending along at least a portion of the shank. The shank may have a leading end proximate the distal tip and a trailing end proximate the head. The screw thread may be continuously variable between a first pitch relatively closer to the trailing end, and a second pitch, larger than the first pitch, relatively closer to the leading end. The method may further include driving rotation of the subtalar arthroereisis screw with the tool to urge advancement of the subtalar arthroereisis screw into the implantation site.

The screw thread may taper continuously from a first major diameter at the trailing end, to a second major diameter, smaller than the first major diameter. Driving rotation of the subtalar arthroereisis screw may include engaging the soft tissues with the screw thread.

The screw thread may have a plurality of notches distributed along its length. Driving rotation of the subtalar arthroereisis screw may include engaging the soft tissues with the notches.

The distal tip may have a domelike shape. Urging advancement of the subtalar arthroereisis screw into the implantation site may include engaging the soft tissues with the domelike shape.

The distal tip may have a generally hemispherical shape. Urging advancement of the subtalar arthroereisis screw into the implantation site may include engaging the soft tissues with the generally hemispherical shape.

The head may be enlarged relative to the shank. The method may further include engaging the soft tissues with the head.

The receiving end of the head may have a drive feature and threading adjacent to the drive feature. The method may further include mating the drive feature with a corresponding drive feature of the tool, and mating the threading with at least one selection from the group consisting of the tool, and a removal tool configured to remove the subtalar arthroereisis screw from the implantation site.

The subtalar arthroereisis screw may have a bore extending through the subtalar arthroereisis screw, from the head to the distal tip. The drive feature may include an interior feature defining a first portion of the bore. The threading may be interior threading distal to the drive feature, defining a second portion of the bore. The method may further include anchoring a K-wire in the implantation site and inserting a proximal end of the K-wire into the bore. Urging advancement of the subtalar arthroereisis screw into the implantation site may include urging advancement of the subtalar arthroereisis screw along the K-wire.

The head may have a pair of cutouts positioned on opposite sides of the head. The method may further include, after urging advancement of the subtalar arthroereisis screw into the implantation site, gripping the head with a removal tool.

The removal tool may be a Kocher clamp.

Each of the cutouts may have a concave surface flanked by two flattened surfaces. According to some embodiments, a subtalar arthroereisis screw may have a head and a body with a distal tip, a shank extending from the head to the distal tip, and a screw thread extending along at least a portion of the shank. The shank may have a leading end proximate the distal tip and a trailing end proximate the head. The screw thread may taper continuously from a first major diameter at the trailing end, to a second major diameter, smaller than the first major diameter. The screw thread may have a plurality of notches distributed along its length. The head may have a receiving end that is connectable to a tool to facilitate rotation of the head with the tool during implantation of the subtalar arthroereisis screw.

The distal tip may have a domelike shape.

The distal tip may have a generally hemispherical shape.

The head may be enlarged relative to the shank.

The receiving end of the head may have a drive feature that mates with a corresponding drive feature of the tool, and threading adjacent to the drive feature and matable with at least one selection from the group consisting of the tool, and a removal tool configured to remove the subtalar arthroereisis screw from an implantation site.

The subtalar arthroereisis screw may have a bore extending through the subtalar arthroereisis screw, from the head to the distal tip. The drive feature may have an interior feature defining a first portion of the bore. The threading may be interior threading distal to the drive feature, defining a second portion of the bore.

The head may have a pair of cutouts positioned on opposite sides of the head. The cutouts may be shaped to facilitate gripping of the head with a removal tool.

The removal tool may be a Kocher clamp.

Each of the cutouts may have a concave surface flanked by two flattened surfaces.

According to some embodiments, a method for performing subtalar arthroereisis may include obtaining access to an implantation site in soft tissues of a foot and connecting a tool to a receiving end of a head of a subtalar arthroereisis screw. The subtalar arthroereisis screw may have the head and a body with a distal tip, a shank extending from the head to the distal tip, the shank having a leading end proximate the distal tip and a trailing end proximate the head, and a screw thread extending along at least a portion of the shank. The screw thread may taper continuously from a first major diameter at the trailing end, to a second major diameter, smaller than the first major diameter. The thread may have a plurality of notches distributed along its length. The method may further include driving rotation of the subtalar arthroereisis screw with the tool to urge advancement of the subtalar arthroereisis screw into the implantation site, such that the notches engage the soft tissues.

The distal tip may have a domelike shape. Urging advancement of the subtalar arthroereisis screw into the implantation site may include engaging the soft tissues with the domelike shape.

The distal tip may have a generally hemispherical shape. Urging advancement of the subtalar arthroereisis screw into the implantation site may include engaging the soft tissues with the generally hemispherical shape.

The head may be enlarged relative to the shank. The method may further include engaging the soft tissues with the head.

The receiving end of the head may have a drive feature and threading adjacent to the drive feature. The method may further include mating the drive feature with a corresponding drive feature of the tool and mating the threading with at least one selection from the group consisting of the tool, and a removal tool configured to remove the subtalar arthroereisis screw from the implantation site.

The subtalar arthroereisis screw may have a bore extending through the subtalar arthroereisis screw, from the head to the distal tip. The drive feature may have an interior feature defining a first portion of the bore. The threading may be interior threading distal to the drive feature, defining a second portion of the bore. The method may further include anchoring a K-wire in the implantation site and inserting a proximal end of the K-wire into the bore. Urging advancement of the subtalar arthroereisis screw into the implantation site may include urging advancement of the subtalar arthroereisis screw along the K-wire.

The head may have a pair of cutouts positioned on opposite sides of the head. The method may further include, after urging advancement of the subtalar arthroereisis screw into the implantation site, gripping the head with a removal tool.

The removal tool may be Kocher clamp.

Each of the cutouts may have a concave surface flanked by two flattened surfaces.

According to some embodiments, a subtalar arthroereisis screw may have a head with a receiving end that is connectable to a tool to facilitate rotation of the head with the tool during implantation of the subtalar arthroereisis screw and a body. The body may have a distal tip with a domelike shape, a shank extending from the head to the distal tip, and a screw thread extending along at least a portion of the shank. The shank may have a leading end proximate the distal tip and a trailing end proximate the head. The subtalar arthroereisis screw may further have a bore extending through the subtalar arthroereisis screw, from the head to the distal tip. The screw thread may be continuously variable between a first pitch relatively closer to the trailing end, and a second pitch, larger than the first pitch, relatively closer to the leading end. The screw thread may taper continuously from a first major diameter at the trailing end, to a second major diameter, smaller than the first major diameter. The screw thread may have a plurality of notches distributed along its length.

According to some embodiments, a method for performing subtalar arthroereisis may include obtaining access to an implantation site in soft tissues of a foot and connecting a tool to a receiving end of a head of a subtalar arthroereisis screw. The subtalar arthroereisis screw may have the head and a body with a distal tip with a domelike shape, a shank extending from the head to the distal tip, the shank having a leading end proximate the distal tip and a trailing end proximate the head, and a screw thread extending along at least a portion of the shank. The screw thread may be continuously variable between a first pitch relatively closer to the trailing end, and a second pitch, larger than the first pitch, relatively closer to the leading end. The screw thread may taper continuously from a first major diameter at the trailing end, to a second major diameter, smaller than the first major diameter. The screw thread may have a plurality of notches distributed along its length. The subtalar arthroereisis screw may further have a bore extending through the subtalar arthroereisis screw, from the head to the distal tip. The method may further include driving rotation of the subtalar arthroereisis screw with the tool to urge advancement of the subtalar arthroereisis screw into the implantation site, such that the notches engage the soft tissues.

### BRIEF DESCRIPTION OF THE DRAWINGS

Exemplary embodiments of the disclosure will become more fully apparent from the following description and appended claims, taken in conjunction with the accompanying drawings. Understanding that these drawings depict only exemplary embodiments and are, therefore, not to be considered limiting of the scope of the appended claims, the exemplary embodiments of the present disclosure will be described with additional specificity and detail through use of the accompanying drawings in which:

FIG. 1 is a side elevation view of a foot with a screw placed pursuant to an arthroereisis procedure according to one embodiment.

FIGS. 2A, 2B and 2C are perspective, front elevation, and side elevation, views, respectively, of the screw of FIG. 1.

FIGS. 3A and 3B are front and side elevation, section views, respectively, of the screw of FIG. 1.

FIG. 4 is a top view of the screw of FIG. 1.

### DETAILED DESCRIPTION

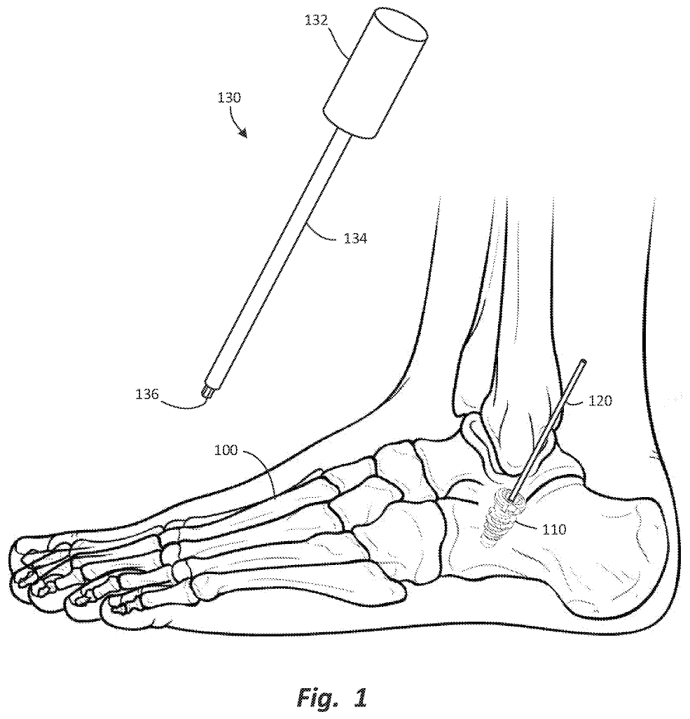

Exemplary embodiments of the disclosure will be best understood by reference to the drawings, wherein like parts are designated by like numerals throughout. It will be readily understood that the components of the disclosure, as generally described and illustrated in the Figures herein, could be arranged and designed in a wide variety of different configurations. Thus, the following more detailed description of the embodiments of the apparatus, system, and method, as represented in FIGS. 1 through 4, is not intended to limit the scope of the claims, as claimed, but is merely representative exemplary of exemplary embodiments.

The phrases “connected to,” “coupled to” and “in communication with” refer to any form of interaction between two or more entities, including mechanical, electrical, magnetic, electromagnetic, fluid, and thermal interaction. Two components may be functionally coupled to each other even though they are not in direct contact with each other. The term “abutting” refers to items that are in direct physical contact with each other, although the items may not necessarily be attached together. The phrase “fluid communication” refers to two features that are connected such that a fluid within one feature is able to pass into the other feature.

The word “exemplary” is used herein to mean “serving as an example, instance, or illustration.” Any embodiment described herein as “exemplary” is not necessarily to be construed as preferred or advantageous over other embodiments. While the various aspects of the embodiments are presented in drawings, the drawings are not necessarily drawn to scale unless specifically indicated.

FIG. 1 is a side elevation view of a human foot, or foot 100, with a screw 110 placed pursuant to an arthroereisis procedure, according to one embodiment. As shown, the screw 110 may be inserted along a K-wire 120.

The surgical procedure may commence with exposure of the operative site and retraction of the surrounding tissues, as needed. The K-wire 120 may then be positioned and inserted into bone under fluoroscopy, at the proper trajectory for insertion of the screw 110. The screw 110 is inserted through the use of any commonly known insertion technique. In some embodiments, the screw 110 may be cannulated so that it has a bore (also referred to as a receiving end; to be shown subsequently) that can receive the K-wire 120. Thus, the proximal end of the K-wire 120 may be inserted into the bore, and then the surgeon may slide the screw 110 along the K-wire 120, into the operative site.

Once the screw 110 has reached the operative site, it may be rotated and driven distally such that it engages the soft tissues of the implantation site. A driver 130 may optionally be used for this purpose. The driver 130 may have a handle 132, a shank 134, and a drive feature 136 at a distal end of the shank 134. The drive feature 136 may be a positive feature (a protrusion) or a negative feature (a recess). As shown, the drive feature 136 may be a hexagonal protrusion sized and shaped to be received in a correspondingly-shaped drive feature of the screw 110 such that the driver 130 can be used to urge the screw 110 to move distally, and can also be used to impart rotation to the screw 110 to cause the threads of the screw 110 to engage the soft tissues.

Once the screw 110 has been fully driven into the soft tissues, the driver 130 and the K-wire 120 may be removed. The surgical site may once again be closed, with the screw 110 in place. The screw 110 may act as a spacer to block the anterior and inferior displacement of the talus, allowing normal subtalar joint motion but blocking excessive pronation and the resulting sequela.

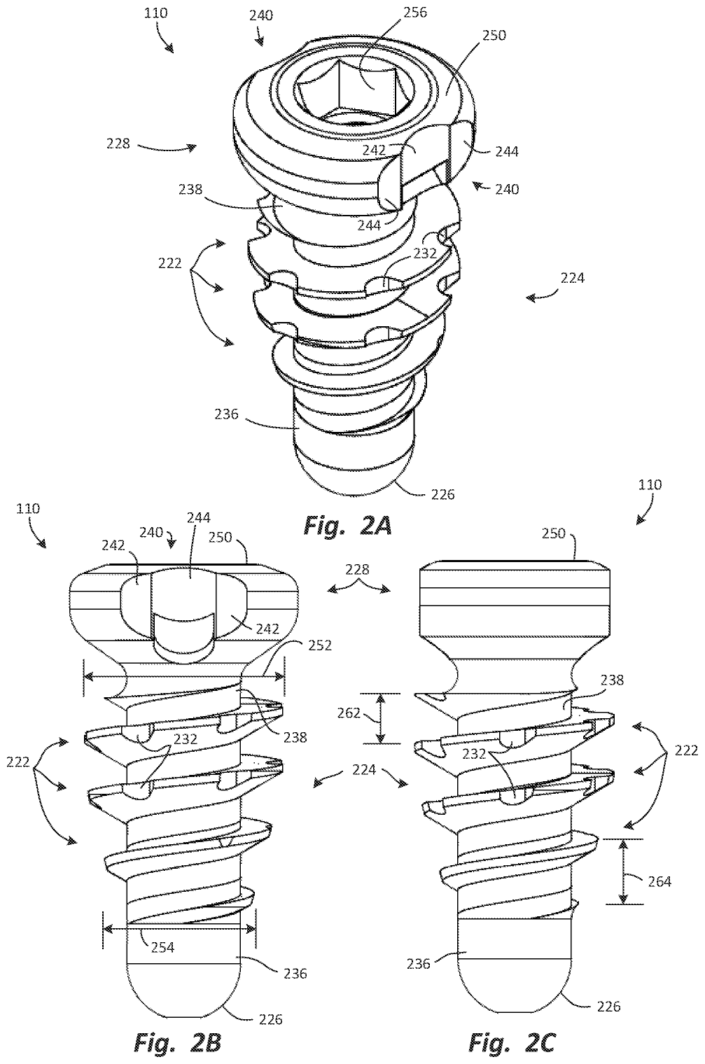

FIGS. 2A, 2B and 2C are perspective, front elevation, and side elevation, views, respectively, of the screw of FIG. 1. These figures show an embodiment in which the screw 110 includes a head 228, a shank 224, and a screw thread 222. A screw according to the present disclosure is not limited to these features but may also include other features and/or elements.

As shown in FIGS. 2A, 2B, and 2C, the shank 224 has a trailing end 238 located proximate, or adjacent to, the head 228, and a leading end 236 proximate to the distal tip 226. The screw thread 222 may be helical and may be positioned to encircle the shank 224. The screw thread 222 tapers in diameter from a first diameter, at which the screw thread 222 has a relatively large surface area (near the trailing end 238) to a second diameter, at which the screw thread 222 has a relatively smaller surface area (near the leading end 236). The screw thread 222 gradually tapers along the shank 224 toward the distal tip 226. By nature of its design, the screw thread 222 may help to facilitate entry of the screw 110 into the tissue of the foot 100 and may also facilitate secure positioning of the screw 110 within the foot 100.

According to this embodiment, the screw 110 may have many features that enable a secure fit within the foot 100 and also enable optimal insertion of the screw 110 into the foot. For example, the distal tip 226 may be domelike in shape to enable smooth and easy entry into the foot. Since the screw 110 may be lodged in soft tissues, rather than bone, the distal tip 226 need not penetrate bone. Thus, the distal tip 226 may be sized such that it is small enough to wedge apart adjacent soft tissues, but also large enough to avoid puncturing such tissues. In the event that the screw 110 is initially positioned improperly in the foot 100, the screw 110 may be withdrawn and placed at a different location and/or orientation, without being drawn into the previous position and orientation by a puncture in the soft tissue. The domelike shape of the distal tip 226 further allows for redirection and reorientation of the anticipated trajectory, the screw threads 222 are engaged in the soft tissue.

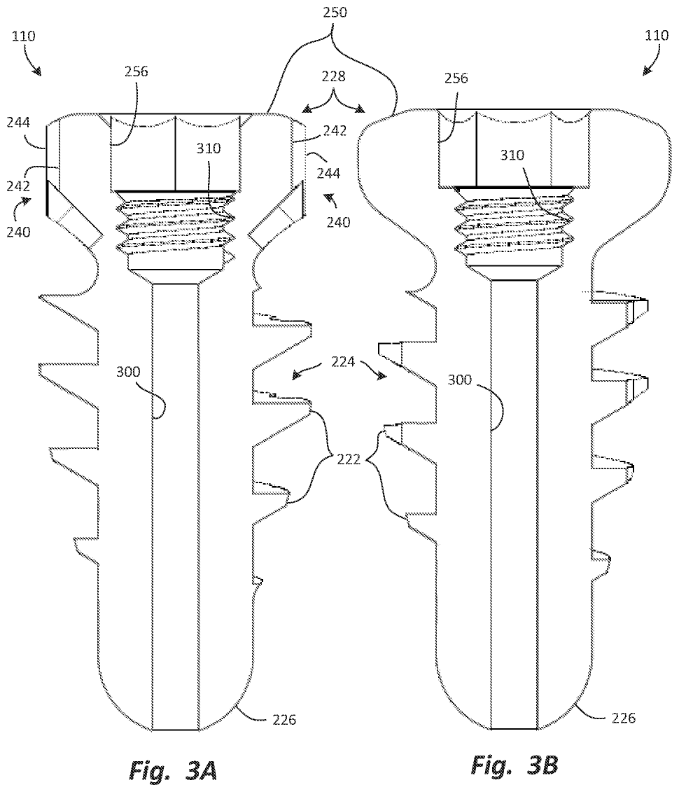

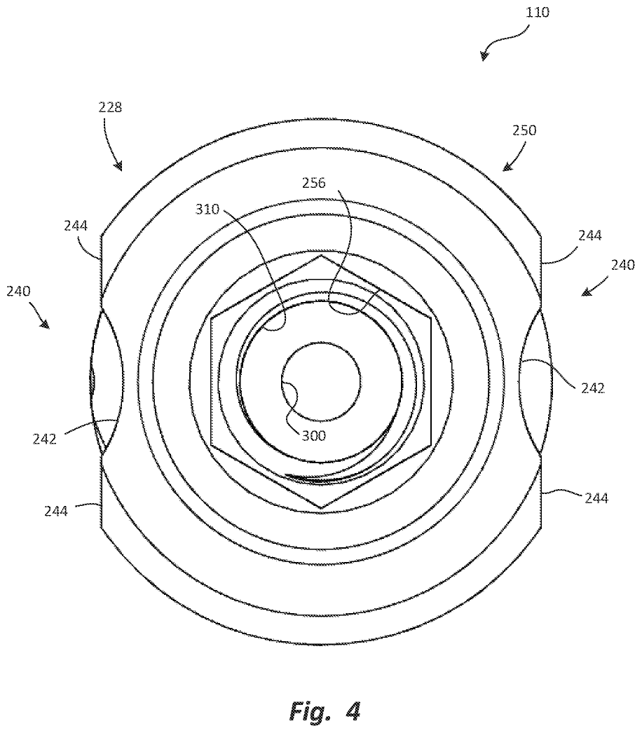

In some embodiments, the distal tip 226 may be not only domelike, but also generally hemispherical in shape. A “generally hemispherical” shape has a radius that is consistent across a volume that defines, or nearly defines, half of a sphere. A “generally hemispherical” shape may thus define a domelike shape with some minor departures from a precise hemispherical shape. For example, the distal tip 226 may be referred to as generally hemispherical even though the intersection of the bore 300 (shown in FIGS. 3A, 3B, and 4) with the distal tip 226 represents a departure from a precisely hemispherical shape.

A generally hemispherical shape may provide benefits beyond those of a shape that is only domelike, but not generally hemispherical, because it may blend more smoothly with the generally cylindrical shape of the leading end 236 of the shank 224. This blending may avoid the existence of a sharp edge between the distal tip 226 and the leading end 236. Such a sharp edge may otherwise catch on and/or unnecessarily injure soft tissues during insertion. Thus, the generally hemispherical shape of the distal tip 226 may ease insertion of the screw 110 into the surgical site and may help to minimize trauma to the surrounding soft tissues during insertion.

Notably, in alternative embodiments, other domelike shapes that are not generally hemispherical may be used to provide similar benefits. Any shape that avoids sharp edges and/or blends gently, at its proximal end, with a cylindrical shape may provide similar advantages. For example, in some embodiments (not shown), an arthroereisis screw may have a distal end with a parabolic shape or the like.

Returning to the screw 110 as shown in FIGS. 2A, 2B, and 2C, at the opposite end of the screw 110 from the distal tip 226 is the head 228. Within the head 228, there is a receiving end 250 connectable to the driver 130. As was more specifically explained above, the driver 130 may have at one end, a drive feature 136 of hexagonal shape to connect to the receiving end 250 of the screw 110, in order to urge the screw 110 to move distally, and impart rotation to the screw 110 causing the screw threads 222 of the screw 110 to engage the soft tissues. The receiving end 250 may have a drive feature 256 that is configured to mate with the drive feature 136 of the driver 130. As embodied in FIG. 2A, the drive feature 256 may be a hexagonal socket shaped to receive the hexagonal protrusion of the drive feature 136. In alternative embodiments, a drive feature may be a positive feature (i.e., a protrusion) rather than a negative feature, and may have any shape suitable for mating with any of various known driver types.

The head 228 may generally be circular in cross-sectional shape but may also have a pair of cutouts 240 positioned on opposite sides of the head 228. The cutouts 240 may define departures from the generally circular cross-sectional shape, that facilitate gripping of the head 228 with a removal device such a pair of forceps. The cutouts 240 may provide an interface with such a removal device that allows rotational motion to be imparted to the screw 110 by the removal device, thereby permitting the removal device to be used to unscrew and remove the screw 110 in a way that minimizes damage to surrounding tissues. In some embodiments, the cutouts 240 may be shaped to receive the ends of a Kocher clamp or the like. Each of the cutouts 240 may optionally define a concave surface 242 flanked by two flattened surfaces 244, as will be shown in greater detail FIG. 4.

As mentioned previously the shape of the screw threads 222 may be configured to facilitate purchase of the screw 110 in the soft tissues of the sinus tarsi. Thus, the screw threads 222 may represent an improvement over the threadforms of many known subtalar screws, which are patterned after those of screws designed to lodge in bone, not soft tissue. In particular, the tapered major diameter, variable pitch, and/or notches 232 of the screw threads 222 may facilitate secure and lasting anchoring of the screw 110 in the foot 100.

Specifically, the screw threads 222 may taper gradually from a first major diameter 252 proximate the head 228, to a second major diameter 254 proximate the distal tip 226 that is significantly smaller than the first major diameter 252. This tapering of the major diameter of the screw threads 222 along the length of the shank 224 may help the screw threads 222 gradually and securely bite into the surrounding tissues during insertion and advancement into the foot 100.

Further, the screw threads 222 may have a variable pitch, with a pitch that is smaller toward the head 228, and larger toward the distal tip 226. As shown, the pitch of the screw threads 222 may taper gradually from a first pitch 262 proximate the head 228 to a second pitch 264 proximate the distal tip 226. The second pitch 264 may be significantly greater than the first pitch 262. This variable pitch may result in compression of the soft tissues as the screw 110 is advanced, as soft tissues are trapped between the adjacent threads of the screw threads 222 and compressed between them as they enter the proximal, lower-pitch portion of the screw threads 222.

Further, there may be a plurality of notches 232 formed in the exterior of the screw thread 222, which can be intermittently distributed lengthwise along the shank 224. The notches 232 in the screw threads 222 can foster the ingrowth of scar tissue, which thereby reduces the likelihood of the screw 110 backing out or otherwise loosening after implantation. Specifically, as the foot 100 heals, the soft tissues in which the screw 110 is lodged may grow scar tissue that enters the notches 232 to help prevent “unscrewing” motion or other motion of the screw 110 within the foot 100.

The tapered major diameter, variable pitch, and/or notches 232 of the screw threads 222 may function synergistically with each other to retain the screw 110 in the soft tissue. For example, the variable pitch may compress soft tissues more tightly between screw threads 222 positioned adjacent to each other as the screw 110 is advanced into the soft tissue. The soft tissue may thus have a tendency to be pushed outward, out of the space between the screw threads 222. This tendency may increase as greater compressive pressure is applied by the screw threads 222 proximate the head 228. The increasing major diameter of the screw threads 222 toward the head 228 may help keep the soft tissues in place, between the screw threads 222, as this compression increases.

As another example, the notches 232 may help counteract the tendency of the screw 110 to back out of its proper position in the surrounding soft tissue, by helping to prevent rotation of the screw 110 after the screw 110 has been implanted. More specifically, soft tissues surrounding the screw 110 may enter the notches 232, thereby tending to block further rotation of the screw 110, in the absence of significant torque (such as the torque imparted to the screw 110 during implantation). The variable pitch, in combination with the notches 232, may increase the rigidity of the surrounding soft tissues due to the compressive effect described previously. As a result, the variable pitch may help the notches lodge in more rigid tissue, increasing the backout resistance of the screw threads 222.

As yet another example, the tapered major diameter of the screw threads 222 may cause the screw threads 222 to gradually bite further into the surrounding soft tissues as the screw 110 is advanced. This gradually intensifying bite may cause the surrounding soft tissues to lodge more firmly in the notches 232. Thus, the presence of the tapered major diameter in combination with the notches 232 of the screw threads 222 may provide greater backout resistance than either feature would accomplish by itself. As set forth above, the same can be said of the tapered major diameter in combination with the variable pitch, and of the variable pitch in combination with the notches 232.

FIGS. 3A and 3B are front and side elevation, section views, respectively, of the screw of FIG. 1. These figures show the interior features of the screw 110. Specifically, the screw 110 may have a bore 300 and interior threading 310.

The bore 300 may pass through the length of the screw 110, from the head 228 to the distal tip 226. The bore 300 may optionally have a circular cross-sectional shape so that the bore 300 can receive a cylindrical K-wire, such as the K-wire 120 shown in FIG. 1. The proximal end of the K-wire 120 may be inserted into the bore 300 at the distal tip 226, and the screw 110 may be slid along the K-wire 120 such that the proximal end of the K-wire 120 emerges from the bore 300 at the head 228. The screw 110 may be further urged (for example, by the driver 130) to slide along the length of the K-wire 120 and to the operative site.

The interior threading 310 may be positioned distal to and adjacent to the drive feature 256 of the head 228. The interior threading 310 may be useful for more secure attachment of an instrument, such as the driver 130 and/or a device (not shown), to the screw 110.

In some embodiments, the interior threading 310 may serve as part of all of a removal tool interface that facilitates connection of the screw 110 to a removal tool (not shown) that can be used to remove or reposition the screw 110. In some embodiments, the driver 130 may operate as a removal tool, and may have a feature (such as threading) that engages the interior threading 310. More specifically, the driver 130 and/or other removal tool may have a threaded tip instead of a polygonal protrusion. In alternative embodiments, the driver 130 and/or the removal tool may have an interior threaded boss that can deploy from within a polygonal protrusion to rotate into engagement with the interior threading 310 for more secure attachment. The interior threading 310 is optional, as in some embodiments, the driver 130 may rely only on engagement with the drive feature 256 of the head 228, and a removal tool may engage only the cutouts 240 and not the interior features of the head 228.

FIG. 4 is a top view of the screw of FIG. 1. The shapes of the cutouts 240 are more clearly shown. The concave surfaces 242 may receive corresponding convex protrusions on a removal device, such as a Kocher clamp. Further, the flattened surfaces 244 may abut corresponding flattened surfaces of the removal tool in order to facilitate transfer of torque from the removal tool to the head 228 to remove the screw 110.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified.

Reference throughout this specification to “an embodiment” or “the embodiment” means that a particular feature, structure or characteristic described in connection with that embodiment is included in at least one embodiment. Thus, the quoted phrases, or variations thereof, as recited throughout this specification are not necessarily all referring to the same embodiment.

Similarly, it should be appreciated that in the above description of embodiments, various features are sometimes grouped together in a single embodiment, Figure, or description thereof for the purpose of streamlining the disclosure. This method of disclosure, however, is not to be interpreted as reflecting an intention that any claim require more features than those expressly recited in that claim. Rather, as the following claims reflect, inventive aspects lie in a combination of fewer than all features of any single foregoing disclosed embodiment. Thus, the claims following this Detailed Description are hereby expressly incorporated into this Detailed Description, with each claim standing on its own as a separate embodiment. This disclosure includes all permutations of the independent claims with their dependent claims.

Recitation in the claims of the term “first” with respect to a feature or element does not necessarily imply the existence of a second or additional such feature or element. Elements recited in means-plus-function format are intended to be construed in accordance with 35 U.S.C. § 112 Para. 6. It will be apparent to those having skill in the art that changes may be made to the details of the above-described embodiments without departing from the underlying principles set forth herein.

While specific embodiments and applications of the present disclosure have been illustrated and described, it is to be understood that the scope of the appended claims is not limited to the precise configuration and components disclosed herein. Various modifications, changes, and variations which will be apparent to those skilled in the art may be made in the arrangement, operation, and details of the methods and systems disclosed herein.

1. A subtalar arthroereisis screw comprising:
a head; and
a body comprising:
a distal tip;
a shank extending from the head to the distal tip, the shank having a leading end proximate the distal tip and a trailing end proximate the head; and
a screw thread extending along at least a portion of the shank;
wherein:
the screw thread is continuously variable between a first pitch relatively closer to the trailing end, and a second pitch, larger than the first pitch, relatively closer to the leading end; and
the head comprises a receiving end that is connectable to a tool to facilitate rotation of the head with the tool during implantation of the subtalar arthroereisis screw.

2. The subtalar arthroereisis screw of claim 1, wherein the screw thread tapers continuously from a first major diameter at the trailing end, to a second major diameter, smaller than the first major diameter.

3. The subtalar arthroereisis screw of claim 1, wherein the screw thread comprises a plurality of notches distributed along its length.

4. The subtalar arthroereisis screw of claim 1, wherein the distal tip comprises a domelike shape.

5. The subtalar arthroereisis screw of claim 4, wherein the distal tip comprises a generally hemispherical shape.

6. The subtalar arthroereisis screw of claim 1, wherein the head is enlarged relative to the shank.

7. The subtalar arthroereisis screw of claim 1, wherein the receiving end of the head comprises:
a drive feature that mates with a corresponding drive feature of the tool; and
threading adjacent to the drive feature and matable with at least one selection from the group consisting of the tool, and a removal tool configured to remove the subtalar arthroereisis screw from an implantation site.

8. The subtalar arthroereisis screw of claim 7, wherein:
the subtalar arthroereisis screw comprises a bore extending through the subtalar arthroereisis screw, from the head to the distal tip;
the drive feature comprises an interior feature defining a first portion of the bore; and
the threading comprises interior threading distal to the drive feature, defining a second portion of the bore.

9. The subtalar arthroereisis screw of claim 1, wherein:
the head comprises a pair of cutouts positioned on opposite sides of the head; and
the cutouts are shaped to facilitate gripping of the head with a removal tool.

10. The subtalar arthroereisis screw of claim 9, wherein the removal tool comprises a Kocher clamp.

11. The subtalar arthroereisis screw of claim 9, wherein each of the cutouts comprises a concave surface flanked by two flattened surfaces.

12-22. (canceled)

23. A subtalar arthroereisis screw comprising:
a head; and
a body comprising:
a distal tip;
a shank extending from the head to the distal tip, the shank having a leading end proximate the distal tip and a trailing end proximate the head; and
a screw thread extending along at least a portion of the shank;
wherein:
the screw thread tapers continuously from a first major diameter at the trailing end, to a second major diameter, smaller than the first major diameter;
the screw thread comprising a plurality of notches distributed along its length; and
the head comprises a receiving end that is connectable to a tool to facilitate rotation of the head with the tool during implantation of the subtalar arthroereisis screw.

24. The subtalar arthroereisis screw of claim 23, wherein the distal tip comprises a domelike shape.

25. The subtalar arthroereisis screw of claim 24, wherein the distal tip comprises a generally hemispherical shape.

26. The subtalar arthroereisis screw of claim 23, wherein the head is enlarged relative to the shank.

27. The subtalar arthroereisis screw of claim 23, wherein the receiving end of the head comprises:
a drive feature that mates with a corresponding drive feature of the tool; and
threading adjacent to the drive feature and matable with at least one selection from the group consisting of the tool, and a removal tool configured to remove the subtalar arthroereisis screw from an implantation site.

28. The subtalar arthroereisis screw of claim 27, wherein:
the subtalar arthroereisis screw comprises a bore extending through the subtalar arthroereisis screw, from the head to the distal tip;
the drive feature comprises an interior feature defining a first portion of the bore; and
the threading comprises interior threading distal to the drive feature, defining a second portion of the bore.

29. The subtalar arthroereisis screw of claim 23, wherein:
the head comprises a pair of cutouts positioned on opposite sides of the head;
the cutouts are shaped to facilitate gripping of the head with a removal tool; and
the removal tool comprises a Kocher clamp.

30. (canceled)

31. The subtalar arthroereisis screw of claim 23, wherein:
the head comprises a pair of cutouts positioned on opposite sides of the head;
the cutouts are shaped to facilitate gripping of the head with a removal tool; and
each of the cutouts comprises a concave surface flanked by two flattened surfaces.

32-40. (canceled)

41. A subtalar arthroereisis screw comprising:
a head comprising a receiving end that is connectable to a tool to facilitate rotation of the head with the tool during implantation of the subtalar arthroereisis screw;
a body comprising:
a distal tip comprising a domelike shape;
a shank extending from the head to the distal tip, the shank having a leading end proximate the distal tip and a trailing end proximate the head; and
a screw thread extending along at least a portion of the shank; and
a bore extending through the subtalar arthroereisis screw, from the head to the distal tip;
wherein:
the screw thread is continuously variable between a first pitch relatively closer to the trailing end, and a second pitch, larger than the first pitch, relatively closer to the leading end;
the screw thread tapers continuously from a first major diameter at the trailing end, to a second major diameter, smaller than the first major diameter; and
the screw thread comprises a plurality of notches distributed along its length.

42. (canceled)
